(19) United States
(12) Patent Application Publication
(10) Pub. No.: US 2024/0023813 A1
(43) Pub. Date: Jan. 25, 2024

(54) SYSTEMS AND METHODS FOR ULTRASOUND AND PHOTOACOUSTIC GUIDANCE OF CORONARY PROCEDURES

(71) Applicant: RESEARCH DEVELOPMENT FOUNDATION, Carson City, NV (US)

(72) Inventors: Thomas E. MILNER, Irvine, CA (US); Marc D. FELDMAN, San Antonio, TX (US); Nitesh KATTA, Irvine, CA (US)

(73) Assignee: RESEARCH DEVELOPMENT FOUNDATION, Carson City, NV (US)

(21) Appl. No.: 18/044,408

(22) PCT Filed: Sep. 14, 2021
(86) PCT No.: PCT/US21/50147

§ 371 Date: Mar. 8, 2023

Related U.S. Application Data
(60) Provisional application No. 63/078,096, filed on Sep. 14, 2020.

(51) Int. Cl. A61B 8/08; A61B 5/00; A61B 8/00

(52) U.S. Cl. CPC A61B 8/0891; A61B 5/0095; A61B 5/6852; A61B 8/4461; A61B 8/4488

(57) ABSTRACT

Apparatus, systems and methods for ultrasound and photoacoustic guidance of coronary procedures are disclosed herein. Certain embodiments include a first catheter comprising an ultrasound transceiver, and a second catheter comprising a proximal end and a distal end, with a photoacoustic excitation light transmitter positioned at the distal end of the second catheter. The photoacoustic excitation light transmitter can be configured to emit excitation light in a conical pattern and at a specific pulse duration. The second catheter can be configured to detect photoacoustic signals resulting from the absorption of excitation light emitted by the photoacoustic excitation light transmitter.

### CROSS-REFERENCE TO RELATED APPLICATIONS

This application claims priority to U.S. Provisional Patent Application 63/078,096, filed Sep. 14, 2020, the entire contents of which are incorporated by reference herein.

### BACKGROUND INFORMATION

Coronary artery atherosclerosis is the most common type of cardiovascular disease and results in the death of 370,000 American's each year. Although percutaneous coronary intervention (PCI) procedures that utilize endovascular devices and stenting have provided an effective and minimally invasive treatment option for many patients with severe coronary artery atherosclerosis, their use for treating chronic total occlusions (CTO) remains difficult and controversial. CTOs are found in twenty percent of patients undergoing angiography who have coronary artery atherosclerosis [1] and are associated with increased morbidity and mortality [2, 3] because existing treatment options are suboptimal. Current CTO treatment options include coronary artery bypass graft or CABG (thirty percent), management with pharmaceuticals (sixty percent), and PCI (ten percent) [1, 4]. Although CABG is effective in decreasing morbidity and mortality, the procedure is expensive, impractical for elderly or frail patients and requires relatively long recovery times. Another important limitation of CABG is that the majority of bypass grafts utilize veins, which fail prematurely due to the re-purposing of venous grafts from a low- to high-pressure environment. Increased arterial pressures in re-purposed venous grafts result in persistent endothelial cell injury and premature development of atherosclerosis. As a result, 50% of venous grafts become occluded within ten years following CABG.

CTO management with pharmaceuticals does not treat underlying disease mechanisms and often fails to decrease number of major adverse cardiac events (MACE) and offers only minimal improvement in patient outcome. PCI, on the other hand, has been shown to significantly improve patient outcomes by reducing angina and MACE [5, 6]. Despite being highly effective and minimally invasive, PCI is only used in ten percent of CTO cases because these procedures are complex, technically difficult, and only a small number of interventional cardiologists are capable of performing them [4]. The difficulty in treating CTOs with PCI are challenges associated with navigating a steerable wire to cross the hard fibro-calcific material comprising the lesion. Current CTO crossing methods include advancing a wire around the plaque and into the subintimal space in the vessel wall and re-entering the true lumen. Risks of subintimal crossing include vessel wall perforation and side branch occlusion, resulting in tamponade and myocardial infarction. Moreover, in subintimal crossing, final stenting inside the vessel wall with dissection re-entry is associated with increased restenosis and stent thrombosis, compared to true-lumen CTO crossing and stenting [7-11].

Conventional PCI procedures used for non-CTO stenotic lesions in coronary arteries involve navigating a wire through the narrowed lumen of the affected artery [12]. Once the wire is successfully navigated across the atherosclerotic plaque, a stent or atherectomy device can be deployed to further open the narrow passage, restoring normal blood flow. When applied to coronary CTOs, these conventional PCI procedures generally fail because the coronary artery is 100% occluded over one-two centimeters in length so that passing a wire through the CTO while remaining true-lumen is nearly impossible for most interventionalists. Challenges with true-lumen PCI procedures include safely piercing through the hard fibro-calcific CTO cap while simultaneously avoiding unintended mis-direction of the wire into the subintimal space risking vessel wall perforation and loss of arterial side branches.

An alternative method endeavors to purposely direct the wire to the side of the hard fibro-calcific CTO cap, a procedure known as subintimal crossing. Subintimal crossing methods involve advancing a wire through the vessel wall surrounding the CTO lesion. The vessel wall is a three-layered structure comprised of a thin endothelial layer (intima), a middle layer (media) and an outer layer. Atherosclerotic disease thickens all three layers. In subintimal crossing, the wire is looped and purposely advanced past the CTO in the space between the atherosclerotic plaque and outer layers of the vessel wall from either an antegrade and/or retrograde direction. After advancing the wire through the vessel wall past the CTO, a stent is deployed within the vessel wall or subintimal space to re-establish blood flow. Once past the CTO, the wire must be redirected from the subintimal space back into the true-lumen of the coronary artery. Because safely re-directing the wire back into the true-lumen is a difficult step, specialized devices are utilized such as Stingray balloons and knuckle wires which may spontaneously re-enter at vessel bifurcations.

While pharmacologic applications can be used to treat angina that patients with CTOs suffer from, these are not always successful in the elimination of angina symptoms. Medical interventional procedures are used to provide CTO treatment in cases where medication is not successful. The two primary existing procedures for addressing CTOs include coronary artery bypass grafting (CABG) and percutaneous coronary intervention (PCI). CABG is an invasive surgical procedure in which a healthy artery or vein is grafted, past the occluded coronary artery. The grafted vessel bypasses the occluded region of the coronary artery and provides a path for blood to flow to the heart muscle. CABG is a relatively expensive procedure, more traumatic to the patient with longer patient recovery times as compared to PCI.

PCI procedures typically include advancing a collapsed stent into the occluded region and expanding the stent to provide a passageway through the previously-blocked vessel. Such procedures generally involve directing a guide wire through the occluded region to allow for placement of a stent. However, in the case of CTOs, it is often not possible to direct the guide wire through the occluded region which is completely blocked. In such cases, treatment options include directing a guide wire or other components through or around the occlusion, including through the vessel wall. Specifically, many typical treatment options require directing components through the subintimal space in the vessel.

Such treatment options present significant challenges to the physician, due in part to the limited space available and associated risks presented. One of the primary challenges is directing a mechanical component past the occlusion without inadvertently perforating the vessel wall. Such a perforating of the vessel wall can lead to severe complications, such as cardiac tamponade, and it is therefore highly desirable to avoid such risks when treating patients with vascular occlusions.

In addition, such treatment options can require significant amounts of time to perform—typically up to three hours. Completing a procedure requires extreme concentration by the physician over prolonged time periods while performing extremely precise maneuvers. This can lead to physician fatigue and increase the risks of inadvertently perforating the vessel wall. Accordingly, few cardiologists are capable of, or willing to, perform PCI procedures of CTOs. The extended time periods for such procedures also result in redirection of resources (both equipment and human) normally available for other procedures.

Due to the technical difficulty and procedural complexity of subintimal crossing, only a small number of highly specialized interventional cardiologists perform the procedures. Furthermore, from the day subintimal crossing was first reported by Antonio Columbo, MD in 2003 to the present the procedure remains controversial due to increased risk of serious complications such as vessel wall perforation [13, 14]. Vessel wall perforation results in rapid loss of blood into the pericardial sac which normally results in the patient falling into shock. Rapid blood loss must be immediately stopped either through intravascular placement of a metallic coil or deploying a covered stent at the perforation site. Placement of a metallic coil induces a localized clot that stops bleeding but results in a down-stream infarct. Deploying a covered stent against the vessel wall can seal the perforation but has a high rate of vessel restenosis. Another serious risk associated with subintimal crossing PCI procedures is side branch perforation and/or occlusion. During subintimal crossing, the wire can encounter and perforate arterial side branches resulting in myocardial infarction.

Alternatively, when a stent is deployed in the sub-intimal space to restore blood flow to the coronary artery, side-branches can become occluded by stent expansion as shown in FIG. 1 Panels B and C. This is a common complication of branch closure during ballooning, where a CTO subintimal crossing in one artery causes blood flow stoppage in a side branch.

These serious risks and potential complications together with the requirement for highly specialized interventional cardiologists have limited application of subintimal crossing procedures such that only approximately ten percent of CTO patients receive the benefits of a PCI treatment.

Although most patients with calcified CTOs undergoing PCI are currently treated using the subintimal crossing procedure, recent studies demonstrate that true-lumen approaches actually lead to better patient outcomes [15]. Restenosis rates, stent thrombosis rates, and patient mortality are improved with true-lumen approaches versus subintimal crossing [15]. As a result, many intravascular devices that are utilized to perform true-lumen CTO crossing have been developed and tested clinically [16]. These intravascular devices include optical fiber-delivered excimer or mid-infrared laser radiation for CTO ablation, high frequency ultrasound (HIFU), acoustic wires, wire centering devices, and mechanical rotablators with sideview OCT imaging. All of these devices have failed in clinical testing for crossing coronary artery CTOs [16]. Excimer laser devices have failed in coronary arteries due to inefficient ablation of calcium, poor guidance and non-specific residual thermal damage. Although mid-infrared laser devices can readily cut through hard fibro-calcific CTO caps [17], these devices have failed due to poor guidance and non-specific thermal injury. HIFU devices have failed due to non-specific thermal damage and large device size for the most distal coronary arterial segments. Acoustic wires have failed because bends in the wire needed to navigate a tortuous coronary artery lead to unwanted contact with the vascular wall delivering non-specific acoustic energy resulting in vessel injury [16]. Wire centering devices do not assist at piercing the calcified fibrous cap. Rigid mechanical cutting devices such as rotablators with sideway viewing OCT for the peripheral circulation do not have the flexibility to safely navigate tortuous coronary arteries risking vessel wall perforation, and are too large to be used in coronary arteries. Inasmuch as interventional cardiologists do not have readily available access to safe and robust PCI devices capable of true-lumen CTO crossing, an opportunity is recognized to realize a device which can extend the benefits of PCI treatment to the 90% of CTO patients currently treated with pharmaceuticals or CABG.

Accordingly, systems and methods are desired that overcome these and other limitations associated with existing systems and methods.

### SUMMARY

An urgent need is recognized for new endovascular systems and methods that simplify PCI, making the procedure accessible to all interventional cardiologists and increasing number of CTO patients who can be successively treated by PCI rather than CABG or pharmaceuticals. Exemplary embodiments of the present disclosure include systems and methods capable of treating vascular occlusions (e.g. restoring blood flow through the occluded region of the vessel) that address shortcomings of existing treatment options. Particular embodiments include systems and devices which cross in the true-lumen of the vessel.

Prior attempts to develop PCI true-lumen catheter devices and procedures for crossing CTOs in the coronary arteries have included shortcomings that provided obstacles to successful implementation. For example, such systems incorporated over-sized catheter devices that are inflexible or un-steerable. In addition, such systems did not provide real-time guidance and nor incorporate a navigation system. For example, although an optical coherence tomography (OCT) guidance system has been incorporated into a mechanical atherectomy device, the increased size and inflexible tip have limited clinical application of this device to the peripheral arteries in the lower extremities, and not allowed its use in smaller coronary arteries which have increased curvature and tortuosity. When a guidance system is combined with the atherectomy or cutting device, size of the catheter increases and makes steering and navigation more difficult.

A number of potential CTO guidance imaging systems are recognized including ultrasound, photoacoustics, optical coherence tomography (OCT) and x-ray imaging. However, each of these candidate guidance systems includes potential limitations associated with ranging depth, spatial resolution, device size and biological compatibility. Although ultrasound has an excellent ranging depth and is biologically compatible, spatial resolution can be limited by the array size or need for a rotating catheter. Photoacoustics is similar to ultrasound having excellent ranging depth, good biological compatibility and tissue specificity but incorporates both an excitation light source and an ultrasound receiver that can increase catheter size. OCT provides good spatial resolution, but has a limited ranging depth (typically approximately 2 mm) and utilizes a rotating catheter, thus increasing the size of the CTO device. X-ray imaging can meet the requirements for both ranging depth and resolution, as well as place the source outside the body. However, X-ray can be limited in application by biological compatible dosimetry, as well as poor imaging of highly curved and tortuous occluded coronary arteries. Further, only calcium in the arterial wall and luminal plaque allows identification of the coronary artery with X-ray, but adventitial calcium and luminal calcium cannot be differentiated by conventional X-ray approaches.

Exemplary embodiments of the present disclosure include a CTO guidance and treatment system that provides high resolution imaging and a small-sized steerable catheter that uses pulsed laser radiation to cross the CTO. In certain embodiments, the guidance system combines both ultrasound and photo-acoustics using an intra-cardiac echo (ICE) (e.g. placed in either the right atrium or right ventricle) thereby freeing space and reducing size of the crossing-catheter.

Exemplary embodiments employing an ultrasound/photoacoustic guidance system and laser CTO catheter can potentially substantially improve coronary CTO patient outcomes with the successful introduction of a true-lumen PCI approach with a small-sized catheter with excellent guidance capabilities.

Certain embodiments include an apparatus configured for guidance for treatment of a chronic total occlusion, where the apparatus comprises a first catheter comprising an ultrasound transceiver; a second catheter comprising a proximal end and a distal end; and a photoacoustic excitation light transmitter positioned at the distal end of the second catheter, where: the photoacoustic excitation light transmitter emits excitation light in a conical pattern; the photoacoustic excitation light transmitter emits excitation light at a pulse duration between 50 femtoseconds (fs) and 1 microsecond (μs); and the second catheter configured to detect photoacoustic signals resulting from the absorption of excitation light emitted by the photoacoustic excitation light transmitter.

Particular embodiments further comprise a control module, where the control module is coupled to the first catheter and the second catheter. In some embodiments, the ultrasound transceiver is configured as a phased array. In particular embodiments, the ultrasound transceiver comprises a plurality of transducers arranged in a circumferential row extending around the ultrasound transceiver. In certain embodiments, the circumferential row is a first circumferential row, and wherein the plurality of transducers are further arranged in a second circumferential row extending around the ultrasound transceiver. In specific embodiments, the control module is configured to control the pulse duration of the excitation light. In certain embodiments, the second catheter comprises a photonic crystal fiber. In particular embodiments, the photonic crystal fiber is a double clad photonic crystal fiber. In some embodiments, the double clad fiber comprises a core and a cladding, and the photoacoustic excitation light transmitter is configured as a conical tip of the cladding at the distal end of the second catheter. In specific embodiments, the conical tip extends outward from the distal end. In certain embodiments, the conical tip extends inward from the distal end. In particular embodiments, the core is configured to provide illumination for close-range imaging of a region directly in front of the distal end. In some embodiments, the photoacoustic excitation light transmitter is configured as a conical tip of the photonic crystal fiber at the distal end of the second catheter. In certain embodiments, the photonic crystal fiber comprises a multi-faceted tip. In specific embodiments, the second catheter is configured to emit excitation light at a wavelength of 930 nanometers (nm). In certain embodiments, the second catheter is configured to emit excitation light at a wavelength between 1200 nm and 1240 nm. In some embodiments, the second catheter is configured to emit excitation light at a wavelength of 1210 nm. In specific embodiments, the second catheter is configured to emit excitation light at a wavelength between 1700 nm and 1740 nm.

In certain embodiments, the second catheter is configured to emit excitation light at a wavelength of 1720 nm. In particular embodiments, the second catheter is configured to emit excitation light at a first wavelength that is lipid-specific and a second wavelength that is blood-specific. In some embodiments, the second catheter is configured to emit excitation light at a first wavelength of 915 nm, 1210 nm, or 1720 nm and a second wavelength of 532 nm, 980 nm, or 808 nm.

Certain embodiments include a method of imaging a blood vessel containing a chronic total occlusion (CTO), where the method comprises: directing a first catheter into a region of a heart, wherein the first catheter comprises an ultrasound transceiver; directing a second catheter into an artery comprising a chronic total occlusion (CTO); emitting photoacoustic excitation light from a distal end of the second catheter (where the photoacoustic excitation light is emitted in a conical pattern; the photoacoustic excitation light is emitted at a pulse duration between 50 fs and 1 us; and the photoacoustic excitation light generates a photoacoustic signal by light absorption in tissues surrounding the artery or in tissue in the CTO); and detecting the photoacoustic signal emitted from the periphery of the artery or the CTO via first catheter.

In particular embodiments, the artery is a right coronary artery, and the region of the heart where the first catheter is directed is a right atrium proximal to the right coronary artery. In some embodiments, the artery is a left anterior descending artery; and the region of the heart where the first catheter is directed is a right ventricle proximal to the left anterior descending artery. In particular embodiments, the artery is a left anterior descending artery, and the region of the heart where the first catheter is directed is a vein proximal to the left anterior descending artery. In specific embodiments, the artery is a left circumflex artery; and the region of the heart where the first catheter is directed is a right ventricle proximal to the left anterior circumflex artery. In particular embodiments, the artery is a left circumflex artery, and the region of the heart where the first catheter is directed is a vein proximal to the left anterior circumflex artery. In certain embodiments, the artery is a left anterior descending artery; and the region of the heart where the first catheter is directed is a left ventricle proximal to the left anterior descending artery. In particular embodiments, the artery is a left circumflex artery; and the region of the heart where the first catheter is directed is a left ventricle proximal to the left anterior circumflex artery. In certain embodiments, the photoacoustic excitation light is emitted at a wavelength of 930 nanometers (nm). In particular embodiments, the second catheter is configured to emit excitation light at a wavelength between 1200 nm and 1240 nm. In some embodiments, the second catheter is configured to emit excitation light at a wavelength of 1210 nm. In specific embodiments, the second catheter is configured to emit excitation light at a wavelength between 1700 nm and 1740 nm. In certain embodiments, the second catheter is configured to emit excitation light at a wavelength of 1720 nm.

Particular embodiments further comprise: emitting a transmitted ultrasonic signal from the ultrasound transceiver; and receiving a remitted ultrasonic signal by the ultrasound transceiver. In some embodiments, the photoacoustic signal and the remitted ultrasonic signal are utilized to kinematically direct the second catheter. In specific embodiments, the kinematic direction may comprise any combination of mechanical translation or re-orientation. In particular embodiments, the first catheter and the second catheter are coupled to a control module; and the control module is configured to control the pulse duration of the excitation light. In some embodiments, the second catheter comprises a photonic crystal fiber; and the photoacoustic excitation light is emitted from the photonic crystal fiber. In specific embodiments, the photonic crystal fiber is a double clad photonic crystal fiber. In certain embodiments, the double clad fiber comprises a core and a cladding; and the photoacoustic excitation light is emitted from a conical tip of the cladding at the distal end of the second catheter. In particular embodiments, the conical tip extends outward from the distal end of the second catheter. In some embodiments, the conical tip extends inward from the distal end of the second catheter. Specific embodiments further comprise illuminating a region directly in front of the distal end of the second catheter via the core of the double clad fiber.

In the following disclosure, the term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The terms “about” and “approximately” mean, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will be apparent to those skilled in the art from this detailed description.

### BRIEF DESCRIPTION OF THE DRAWINGS

The following drawings form part of the present specification and are included to further demonstrate certain aspects of the present disclosure. The invention may be better understood by reference to one of these drawings in combination with the detailed description of specific embodiments presented herein.

FIG. 1 shows a schematic view of an apparatus according to an exemplary embodiment during use.

FIG. 2 shows a schematic view of a portion of the embodiment of FIG. 1.

FIG. 3. shows a schematic view of a portion of the embodiment of FIG. 1.

FIG. 4 shows a schematic view of a portion of the embodiment of FIG. 1.

FIG. 5 shows schematic and ultrasonic images of exemplary embodiments according to the present disclosure.

FIG. 6 shows schematic and ultrasonic images of exemplary embodiments according to the present disclosure.

FIG. 7 shows images of photoacoustic excitation and phantom CTO blood vessel delineation in an ex vivo porcine heart.

of exemplary embodiments according to the present disclosure.

FIG. 8 shows images of photoacoustic excitation and calcified CTO right coronary artery blood vessel delineation for an ex vivo coronary artery from a human heart.

FIG. 9 shows images of photoacoustic excitation and calcified CTO left anterior descending blood vessel delineation is shown in an ex vivo human heart.

FIG. 10 shows a catheter comprising an inflatable portion according to exemplary embodiments according to the present disclosure.

FIG. 11 shows the embodiment of FIG. 10 during use.

FIG. 12 shows the embodiment of FIG. 10 during use.

FIG. 13 shows a partial section schematic view of an apparatus according to an exemplary embodiment comprising a rotating sensor.

FIG. 14 shows a partial section schematic view of an apparatus according to an exemplary embodiment comprising a circumferential sensor.

FIG. 15 shows a perspective view of an apparatus according to an exemplary embodiment comprising two rows of circumferential sensors.

FIG. 16 shows a partial section view of the embodiment of FIG. 15.

FIG. 17 shows a time reversal algorithm shows a degradation of “circle” wall detection with distance.

FIG. 18 shows experimental results of ultrasound and photo-acoustic imaging of a coronary phantom.

FIG. 19 shows a perspective view of an apparatus according to a first exemplary embodiment comprising a photoacoustic excitation light transmitter fiber with a multi-faceted tip.

FIG. 20 shows a perspective view of an apparatus according to a second exemplary embodiment comprising a photoacoustic excitation light transmitter fiber with a multi-faceted tip.

FIG. 21 shows that modified fiber tip geometry can provide a ring of point output according to exemplary embodiments of the present disclosure.

FIG. 22 illustrates a simulated reconstruction that shows the ability to complete all sides of the circle based on the output of the embodiment of FIG. 21

FIG. 23 shows a comparison between two cases of a contiguous (ring) excitation versus an excitation that is a collection of points to complete the ring.

FIG. 24 shows apparatus used for a transvenous imaging approach for a rabbit model.

FIG. 25 shows results of the transvenous imaging approach for a rabbit model in FIG. 24.

FIG. 26 shows a demonstration of transvenous catheter placement in an in vivo porcine heart according to an exemplary embodiment of the present disclosure.

FIG. 27 shows a schematic view and imaging results according to an exemplary embodiment of the present disclosure.

FIG. 28 shows ultrasound and opto-acoustic images of a porcine heart right coronary artery according to an exemplary embodiment of the present disclosure.

### DETAILED DESCRIPTION OF ILLUSTRATIVE EMBODIMENTS

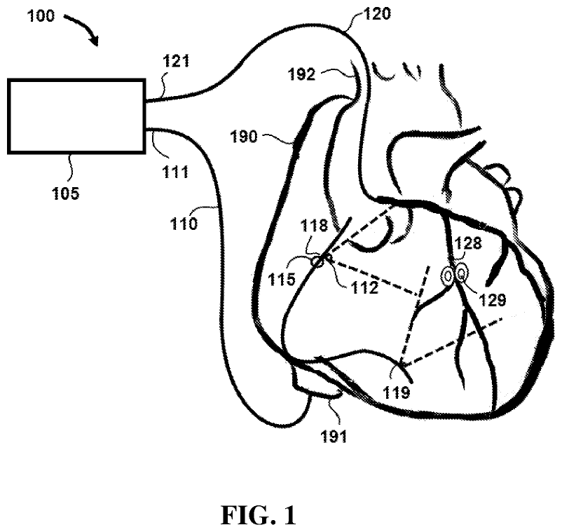

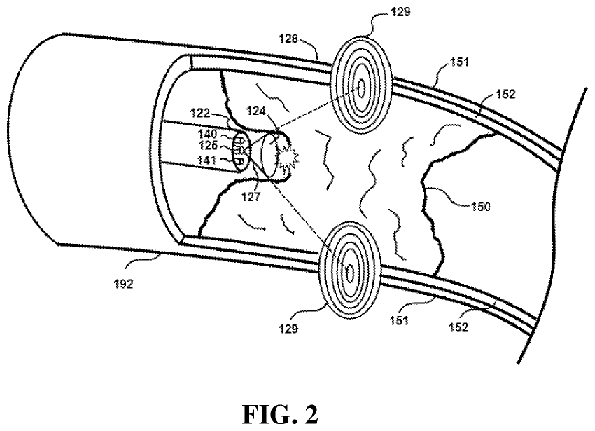

Exemplary embodiments of the present disclosure include systems and methods that utilize for photoacoustic guidance for treatment of a chronic total occlusion in a coronary artery. Referring initially to FIGS. 1-4, an apparatus 100 configured for photoacoustic guidance for treatment of a chronic total occlusion (CTO) 150 is shown. In this embodiment, apparatus 100 comprises first catheter 110 and a second catheter 120 coupled to a control module 105. In certain embodiments, first catheter 110 may be configured as an intracardiac echocardiographic photoacoustic-ultrasound (ICE-PA) detection and second catheter 120 may be configured as a chronic total occlusion (CTO) treatment catheter. FIG. 1 illustrates an overview of a schematic of apparatus 100 during operation with ICE-PA detection catheter 110 inserted into a first artery 191 of a heart 190 and second catheter 120 inserted into a second artery 192 comprising CTO 150. FIGS. 2-4 provide views of specific portions of apparatus 100, as explained in further detail below.

In the embodiment shown, first catheter 110 comprises a proximal end 111 proximal to control module 105 and a distal end 112. ICE-PA first 110 also comprises an ultrasonic transceiver 115 near distal end 112. In addition, second catheter 120 comprises a proximal end 121 coupled control module 105 and a distal end 122. During operation of apparatus 100, distal end 122 can be inserted into an artery of interest, e.g. an artery comprising an occlusion or other condition for treatment. While reference is made to chronic total occlusions in exemplary embodiments discussed herein, it is understood that the visualization and treatment of occlusions or conditions other than chronic total occlusions is also within the scope of the present disclosure. In the illustrated embodiment, a photoacoustic excitation light transmitter 125 is positioned near distal end 122 of second catheter 120. In certain embodiments, first catheter 110 can be placed initially in a desired cardiac location and second catheter 120 can be subsequently directed toward CTO 150, e.g. via a guide catheter (not shown).

As explained in further detail below, first catheter 110 can be inserted into first vessel 191 such that ultrasonic transceiver 115 is placed in location 118 within heart 190. First catheter 110, in particular ultrasonic transceiver 115, can receive photoacoustic signals 129 corresponding with the location of distal end 122 of second catheter 120. These signals can be utilized to assist a user in determining a location of distal end 122 of second catheter 120. First catheter 110 can be placed in different locations depending on which coronary artery contains CTO 150. For example, first catheter 110 can be directed for placement in location 118 in the right atrium or a location 119 right ventricle based on a location 128 of CTO 150. In some examples, first 110 can be placed in location 118 in the right atrium, which is in close proximity to the right coronary artery (RCA) and allows for visualization of second catheter 120 inside the RCA. In other examples, first catheter 110 can be placed in location 119 in the right ventricle to allow visualization of second catheter 120 inside the left circumflex (LCX) and left anterior descending (LAD) arteries. If signal amplitude received by the first catheter is too attenuated for CTOs in the left anterior descending (LAD) or left circumflex (LCx) arteries, the first catheter can be placed in the left ventricle. Closer placement of the first catheter to coronary arteries in the left ventricle will increase photoacoustic and ultrasound signal amplitude.

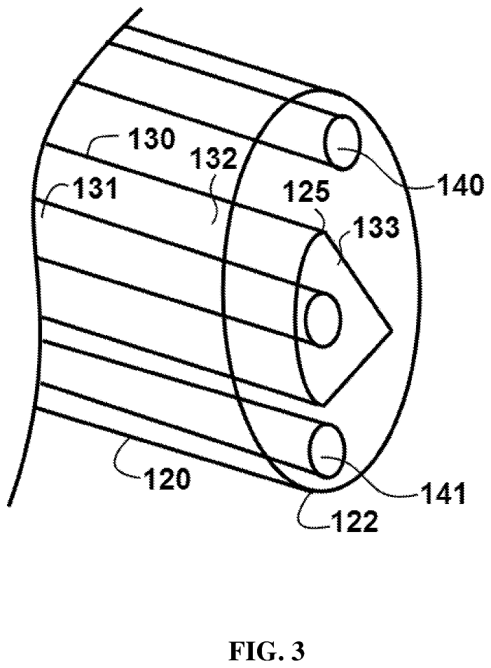

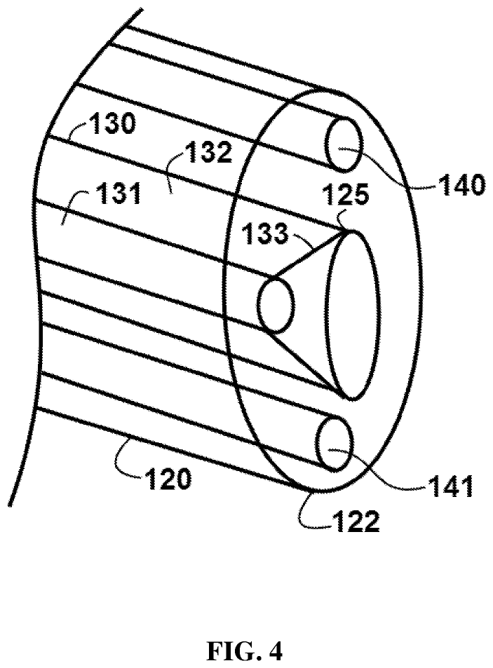

During operation of apparatus 100, photoacoustic excitation light transmitter 125 can emit excitation light 127 in a conical pattern 124 with a particular pulse duration. In particular embodiments, photoacoustic excitation light transmitter 125 is a conical tip of a photonic crystal fiber 130. As shown in FIG. 3 photoacoustic excitation light transmitter 125 can be configured as a conical tip 133 that extends outward from distal end 122. In other embodiments, photoacoustic excitation light transmitter 125 may be configured as a conical tip 133 that extends inward from distal end 122 (e.g. a conical relief formed in distal end 122), as shown in FIG. 4. In particular embodiments, photonic crystal fiber 130 may be a double clad fiber with a core 131 and cladding 131, where conical tip 133 is formed in the fiber cladding 131. In certain embodiments, core 131 may provide illumination for the close-range imaging of the region directly in front of distal end 122.

In certain embodiments, control module 105 controls photoacoustic excitation light transmitter 125 to emit excitation light 127 at a pulse duration between 50 femtoseconds (fs) and 1 microsecond (μs). The photoacoustic signal intensity is determined by the fluence rate (W/m²) of excitation light 127 experienced in the lipid layer and the absorption coefficient at the excitation wavelength. Certain embodiments utilize wavelengths of 930 nm, 1210 nm, 1720 nm for achieving the appropriate fluence rate for strong photoacoustic signal intensity generation to outline the lipid layer given the dosimetry of the radial fiber. Control module 105 maximizes the photoacoustic response by controlling the pulse duration coupled with the pulse energy (mJ pulse energy in nanoseconds vs uJ of pulse energy in picoseconds) First 110 can comprise one or more ultrasonic transceivers 115 configured to detect photoacoustic signals 129 resulting from the absorption of excitation light 127 emitted by the photoacoustic excitation light transmitter 125. In the example shown, photoacoustic signals 129 are generated at the boundary between a lipid layer 151 surrounding the vascular wall 152.

Ultrasonic transceiver 115 can receive photoacoustic signals 129, which can be used to determine a location of photoacoustic excitation light transmitter 125, and consequently, the location of distal end 122 of second catheter 120 with respect to vascular wall 152. The detection of photoacoustic signals 129 can therefore be utilized to assist in treatment (e.g. removal of material from) CTO 150 without perforation of vascular wall 152. In certain embodiments, second catheter 120 may comprise one or more additional lumens 140 and 141 to perform additional functions (e.g. close-range imaging, a vacuum lumen to assist in removal of material, including intermittent or pulsed infusion of saline or CO₂ to transiently cool the CTO and artery in response to pulsed laser irradiation to prevent thermal injury which can lead to later restenosis).

First catheter 110 can be placed in different locations depending on which coronary artery contains CTO 150 and therefore needs to be visualized. For example, first catheter 110 can be directed for placement in a location 118 in the right atrium or a location 119 right ventricle based on a location 128 of CTO 150. In some examples, first catheter 110 can be placed in location 118 in the right atrium, which is in close proximity to the right coronary artery (RCA) and allows for visualization of second catheter 120 inside the RCA. In other examples, first catheter 110 can be placed in location 119 in the right ventricle to allow visualization of second catheter 120 inside the left circumflex (LCX) and left anterior descending (LAD) arteries. Alternatively, first catheter 110 could be placed in the left ventricle to improve imaging of the LAD or LCX.

The radial/conical transmission of excitation light 127 allows for complete visualization of a cross-sectional region of artery 192 in response to a single photoacoustic excitation pulse. Given the high acoustic impedance mismatch between tissue of vascular wall 152 and the fiber 130, ultrasound imaging can determine the location of fiber 130 via ultrasonic transceiver 115. The location of any cross-section of fiber 130 can be identified by maneuvering ultrasonic transceiver 115 of first catheter 110 through rotation and bending to obtain an appropriate cross-sectional or 3D view. In certain embodiments, ultrasonic transceiver 115 can be a phased-array or a single element rotating/tilt ultrasound transducer. Accordingly, photoacoustic excitation from fiber 130 in the lumen of the coronary artery 192 allows for localization and visualization of the CTO structure and the lumen boundary by imaging the lipid layer surrounding the artery—or peri-arterial adipose tissue (PAAT).

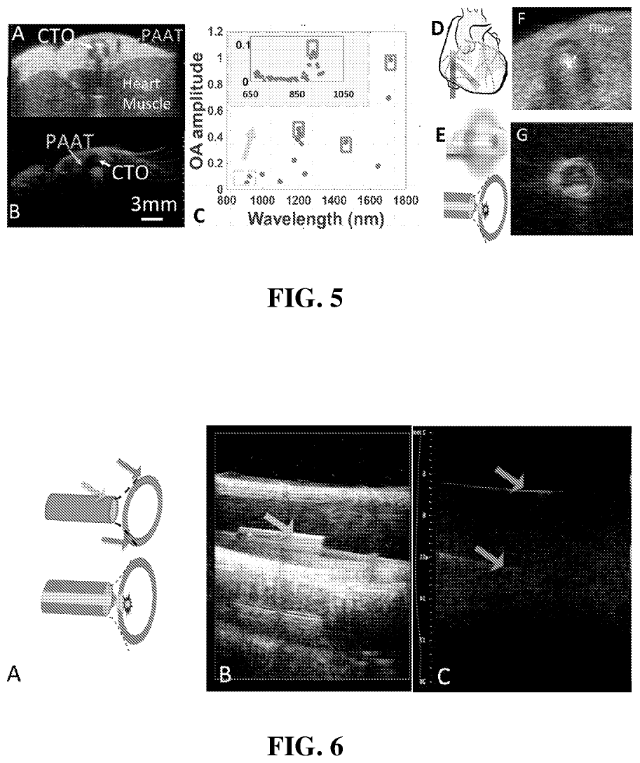

Images acquired by embodiments according to the present disclosure demonstrate successful implementation of the techniques discussed herein. FIG. 5 panel A is an ultrasound (US) image of peri-arterial adipose tissue (PAAT) surrounding an ex vivo human coronary artery with a calcified CTO, while FIG. 5 panel B illustrates photo-acoustic (PA) imaging can delineate blood vessels (BV) even in calcified CTOs. FIG. 5 panel C illustrates the PA signal generated at different wavelengths for excitation light 127. In the embodiment shown in panel C, the PA signal is maximized at 930 nm. In other embodiments, maximal photoacoustic signals can be obtained in the wavelength ranges 1200-1240 nm (peak at 1210 nm), 1700-1740 nm (peak at 1720 nm). FIG. 5 panel D is a schematic overview of concepts discussed in FIGS. 1 and 2, while panel E illustrates a schematic of a conical fiber providing radial illumination from the fiber. FIG. 5 panel F illustrates a phased array ultrasound image of a radial firing fiber inside a diseased ex vivo human coronary artery (“x” indicates the location of the fiber). Finally, FIG. 5 panel G shows PA excitation, imaging and delineation of a diseased ex vivo human BV boundary by an US phased array (simulates ICE-PA) (where the dashed lines delineate the lumen wall).

The upper and lower arrows in the upper schematic diagram of FIG. 6 panel A illustrate forward radial illumination from an optical fiber, which is indicated by the central arrow. The optical fiber includes a glass surface that is shaped to achieve such an illumination spatial profile. The lower schematic diagram in FIG. 6 panel A illustrates a double clad fiber with a central core illuminating tissue directly in front and cladding illuminating a forward conical profile for PA excitation. FIG. 6 panel B shows an ultrasound (US) image that locates the catheter inside a phantom CTO (indicated by arrow). FIG. 6 panel C shows PA imaging can be used to delineate a phantom CTO blood vessel wall (BV), indicated by the arrows in panel C.

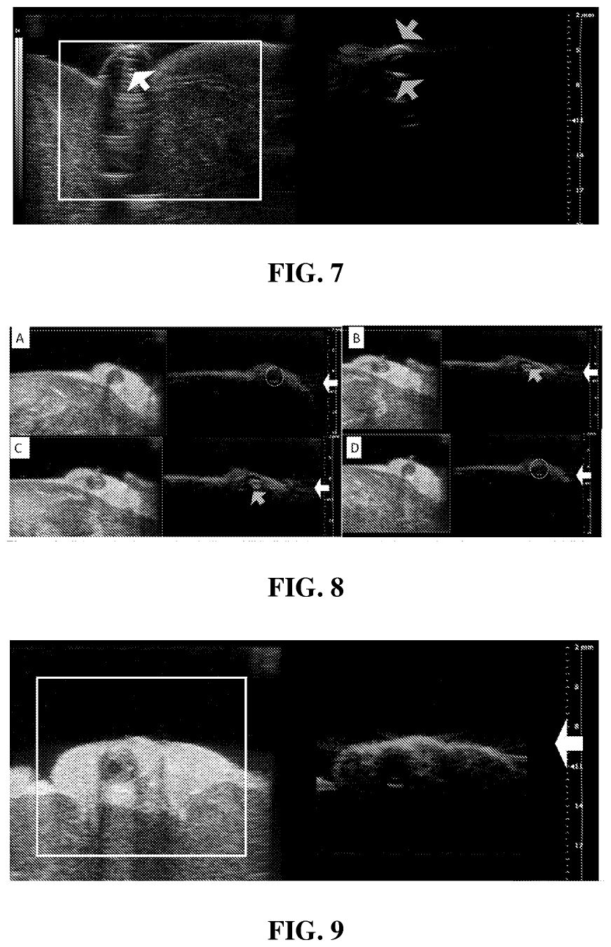

Referring now to FIG. 7, additional results are shown of PA excitation and phantom CTO blood vessel delineation placed in an ex vivo porcine heart. The left portion of FIG. 7 shows US imaging of a phantom CTO blood vessel placed in a porcine heart muscle. The right portion of FIG. 7 shows a PA image of a wall of a phantom CTO blood vessel placed in a porcine heart muscle. The outlined square on the left panel shows the region where the PA image was computed in the right panel. The excitation light (at wavelength 930 nm) in this case is transmitted through a conical illumination fiber (indicated by the arrow in left panel) placed inside the phantom CTO blood vessel illuminating a radial cross-section region of the phantom CTO blood vessel. The two arrows in right panel highlight the contrast delineating the phantom CTO blood vessel wall.

FIG. 8 shows PA excitation and calcified CTO right coronary artery (RCA) blood vessel delineation for an ex vivo coronary artery from a human heart which had previously undergone bypass surgery (CABG). All panels (A,B,C,D) are cross-sectional images of RCA CTO coronary artery as the heart is translated in the field of view of the US/PA imaging system. In panels B and C (unlike panels A and D) lipidic plaque is present inside the CTO (as highlighted in the PA images of Panels B,C by the arrows where signal is seen from inside the coronary artery (i.e. the arrows pointing upward and to the left). Light is focused from outside-in (from the top of the image to 8-11 mm region highlighted in the horizontal arrows pointing to the left in the panels). In each case, the outline of the lumen boundary can be observed clearly (the region is highlighted only in panels A and D with a dashed white circle). The excitation wavelength is selected from the light source in which the highest contrast is obtained in the images. As previously indicated in FIG. 5, excitation wavelengths can be 930 nm, 1200-1240 nm (peak at 1210 nm), 1450-1470 nm, 1700-1740 nm (peak at 1720 nm) (Panel F,G of FIG. 5 are at 1720 nm).

Referring now to FIG. 9, PA excitation and calcified CTO left anterior descending (LAD) blood vessel delineation is shown in an ex vivo human heart. The left portion shows an US image of a LAD blood vessel in the human heart. The right portion presents a PA image (obtained with 930 nm excitation) showing peri-arterial tissue surrounding LAD. Light is focused from outside-in (from the top of the image to 8-11 mm region, highlighted by the arrow to the right of the panel). The outlined square on the left panel shows the region where the PA image was computed in the right panel.

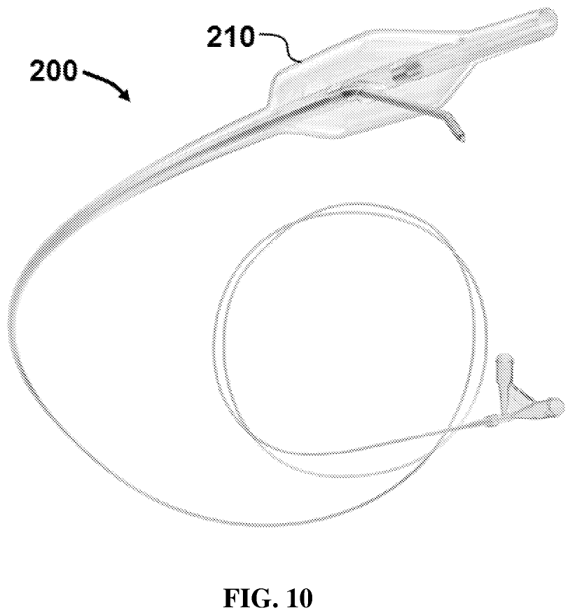

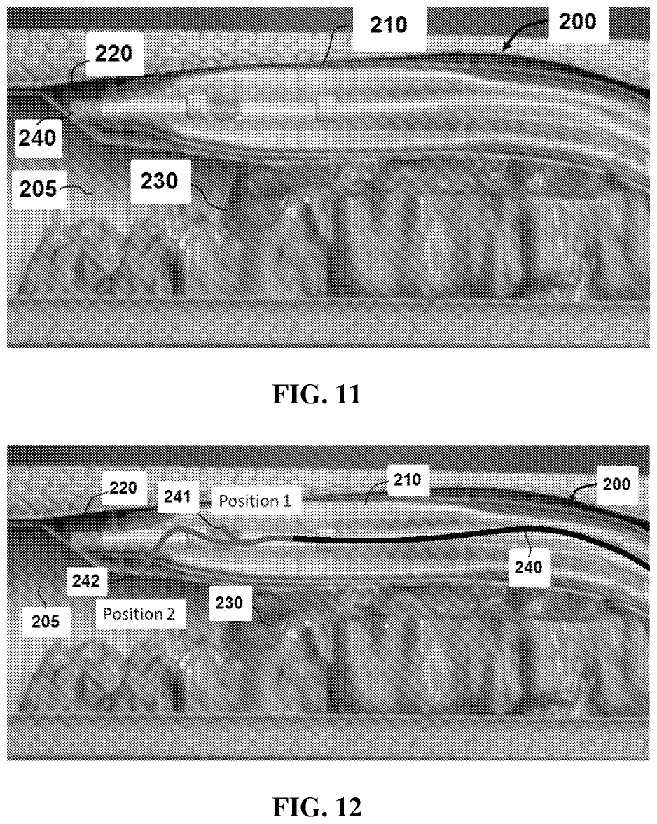

Certain embodiments of the present disclosure may be utilized to assist in other procedures or techniques, including for example, subintimal tracking and re-entry (STAR) crossing for CTOs. Referring now to FIGS. 10-12, STAR techniques utilize a catheter 200 comprising an inflatable flat portion 210. Commercial embodiments of such catheters include the Stingray™ LP coronary system available from Boston Scientific. As shown in FIG. 11, catheter 200 is inserted over a guidewire 240 that has extended past CTO 230 in subintimal space 220 of an arterial blood vessel 205. Catheter 200 is positioned such that flat portion 210 is located within subintimal space 220 proximal to CTO 230.

Flat portion 210 enables a self-orientation along the circumference of arterial blood vessel 205. As shown in FIG. 12, guidewire 240 can exit catheter 200 in a first location 241 located on one side of flat portion 210, or guidewire 240 can exit catheter 200 in a second location 242 located on the opposite side of flat portion 210. In typical STAR procedures, a trial-error procedure is followed in order to locate which side of blood vessel 205 guidewire 240 has to be punctured in order to achieve re-entry into the true-lumen in order restore blood flow.

An optical fiber (guidewire compatible) which can transmit two excitation wavelengths (lipid specific (915 nm, 1210 nm, 1720 nm) and blood specific (532 nm, 980 nm, 808 nm)) can easily locate which side of the re-entry corresponds to blood (Position 2 true-lumen, blood specific) and which side corresponds to the outer wall (Position 1, lipid layer).

However, in certain embodiments of the present disclosure, second catheter 120 may comprise an optical fiber (guidewire compatible) which can transmit two excitation wavelengths that are lipid-specific (e.g. 915 nm, 1210 nm, or 1720 nm) and blood-specific (e.g. 532 nm, 980 nm, or 808 nm). The lipid-specific and blood-specific wavelengths can be used to locate which side of the re-entry corresponds to blood (e.g. location 242 corresponding to the true-lumen) and which side corresponds to the outer wall (e.g. location 241 corresponding to the lipid layer).

In addition, certain embodiments of the present disclosure may be used for guidance in procedures other than CTO treatment. For example, certain embodiments may be incorporated for use in a manner similar to a traditional guidewire, but utilizing OA-ICE principles for guidance rather than X-ray technologies. Such an “optical guidewire” can be implemented, for example, in cardiac catheter intervention (CCI) procedures in distal arteries.

Prior to performing a CCI procedure using traditional techniques, a guide wire is placed into the correct location based on the feedback from traditional X-ray, X-ray computed tomography (CT) and/or X-ray fluoroscopy. The guidewires are typically made of material that contrast with native tissue and can be easily detected in X-ray images. However, such techniques can lead to increased exposure of harmful X-ray radiation to the patient during complicated CCI procedures depending on the coronary arterial network of the patient.

The OA-ICE guidance utilized by embodiments of the present disclosure addresses the problem of X-ray exposure that can be created utilizing traditional techniques. In certain embodiments of the present disclosure, a single optical fiber with a conical tip can act as an optical guidewire instead of the traditional guidewire. Such an optical guidewire can be easily detected in an intracardiac echocardiography image (ICE) due to its high impedance mismatch with native tissue. The opto-acoustic (OA) image generated from this optical guidewire can create contrast generated by blood and peri-arterial adipose tissue (PAAT) typically surrounding coronaries. For example, blood can be imaged via excitation at a wavelength of 532 nm, and PAAT imaged via excitation at a wavelength of 930 nm, 1210 nm, and/or 1720 nm. Such OA-ICE guidance can be used to guide the placement of such an optical guidewire within the anatomy of the coronary arteries.

Once the optical guidewire is placed in the appropriate location, an appropriate CCI tool can be fed along the optical guidewire to reach the location of interest. In certain embodiments, an X-ray contrast marker can also be added to the optical guidewire in order to locate it in the X-ray fluoroscopy field of view as well.

In certain embodiments, the optical guidewire may include an end portion that is shaped conically shaped (as previously discussed in the present disclosure). It is understood that other embodiments may incorporate end portions with different configurations.

During such procedures, an ICE catheter phased array (e.g. a catheter with a photoacoustic excitation light transmitter positioned at the distal end, as described elsewhere in the present disclosure) can be inserted to an appropriate location. In specific embodiments, the location may be a position on the right atrium, right ventricle, or other location that can provide a wide “field-of-view” (e.g. photoacoustic excitation light transmission range) of the heart anatomy and structures of interest. The derived OA-ICE signal can be used to guide the traversal of the “optical guidewire” before performing the desired (e.g. CCI) procedure.

Certain embodiments may also comprise a control system, including for example, an interface that can manipulate the positioning of the optical guidewire (e.g. as defined by the r, theta and z cylindrical coordinates). In particular embodiments, the optical guidewire positioning can be controlled by the interface as the guidewire traverses through the patient's coronary artery network to reach the appropriate region for the desired procedure.

After the optical guidewire is deployed, the appropriate tool (e.g. such as those used in CCI procedures) can be fed around the fiber in a manner similar to current guidewires. In specific embodiments, the optical guidewire can be controlled by a robotic feedback system to automate the guidewire deployment process. Examples of such robotic systems are disclosed in Appl. Sci. 2019, 9(20), 4305; https://doi.org/10.3390/app9204305. In contrast to previously-described embodiments, optical guidewire embodiments do not incorporate features (e.g. a Ho:YAG laser) for traversing a CTO. However, specific examples of optical guidewire embodiments do incorporate lipid/blood excitation light that radiates out in a cone from the optical guidewire and produce OA-ICE signals to assist in guiding a CCI or other appropriate tool to the desired location.

In certain embodiments, it may be desirable to insert first catheter 110 into a vein (instead of e.g. an atrium or ventricle region) that is proximal to an artery comprising CTO 150. For example, if location 128 of CTO 150 (or other condition being treated) is in the left anterior descending (LAD) artery it may be desirable to insert catheter 110 into a vein that is proximal to the LAD artery (e.g., AIV). Similarly, if location 128 is in the left circumflex (CFX) artery, it may be desirable to insert catheter 110 into a vein (e.g., great cardiac vein) that is proximal to the left CFX artery.

The placement of first catheter 110 in a proximal vein may be desirable, for example, when placement in an atrium or ventricle region would not provide sufficient detection sensitivity of the photoacoustic signals 129. The placement of placement of first catheter 110 in a proximal vein may also be desirable, for example, when there is not sufficient space within the artery comprising CTO 150 (or other condition being treated) to insert both first catheter 110 and second catheter 120.

The placement of first catheter 110 into a vein proximal to location 128 can provide certain advantages is such situations. For example, by reducing the distance between location 128 and ultrasonic transceiver 115, the intensity or strength of photoacoustic signals 129 detected by ultrasonic transceiver 115 can be increased. This can increase the accuracy of location 128 detected by first catheter 110. In addition, placement of first catheter 110 in a vein proximal to location 128, but not within the artery being treated, can provide greater space for second catheter 120 to maneuver to clear CTO 150 or otherwise address the condition being treated within the artery.

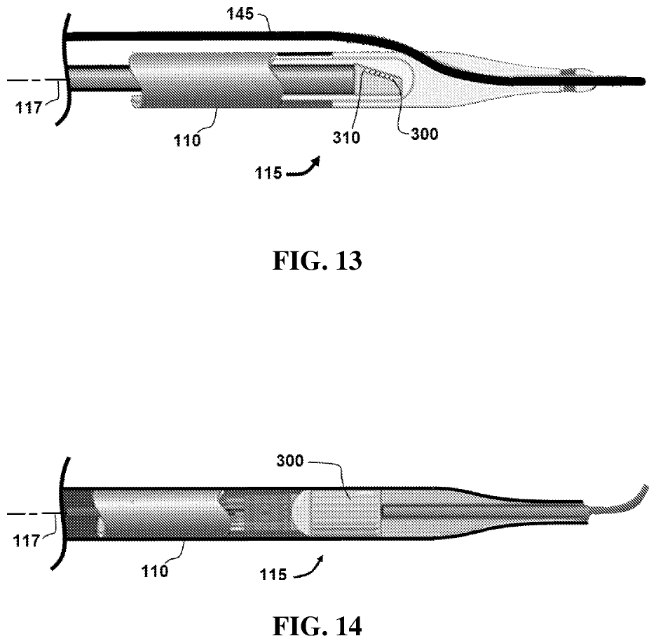

Certain embodiments may also comprise different configurations of ultrasonic transceiver 115. In particular, certain embodiments of ultrasonic transceiver 115 may comprise one (or more) transducers that rotate during use to scan the surrounding environment. Other embodiments of ultrasonic transceiver 115 may comprise an array of transducers extending around the outer circumference or periphery of ultrasonic transceiver 115. FIGS. 13 and 14 illustrate embodiments of ultrasonic transceiver 115 comprising a rotating sensor and a circumferential sensor, respectively. In the embodiment shown in FIG. 13, a linear array 310 of transducers 300 are shown. During operation of the embodiment shown in FIG. 13, array 310 is rotated about a central axis 117 of first catheter 110. By rotating array 310 about axis 117, transducers 300 are capable of transmitting and/or receiving photoacoustic signals around the entire circumference of a lumen (e.g. an artery or vein) into which first catheter 110 has been inserted. In the embodiment shown, a guide wire 145 is used to insert first catheter 110. Embodiments utilizing a guide wire and a rotating sensor array can generate artifacts due to the guide wire (e.g. the guide wire may restrict photoacoustic signals from being transmitted or received by transducers 300 of linear array 310 during a portion of the rotation).

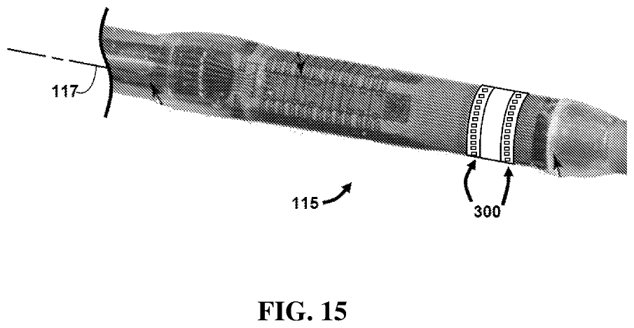

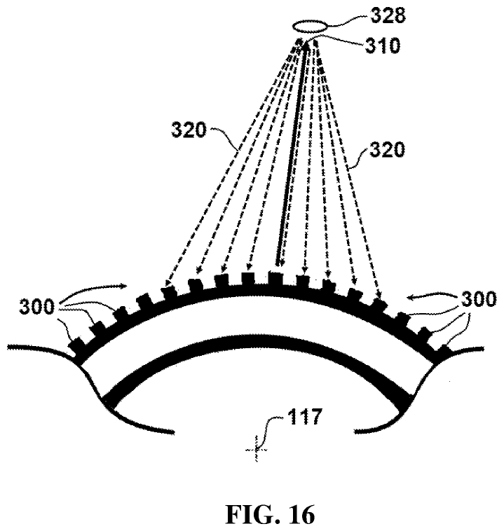

Referring now to FIGS. 15-16, a specific embodiment of ultrasonic transceiver 115 comprises a plurality of transducers 300 extending around the perimeter of ultrasonic transceiver 115. A perspective view is shown in FIG. 15, and a partial section view of ultrasonic transceiver 115 during use is shown in FIG. 16. In this embodiment, transducers 300 are arranged in a first circumferential row 301 and a second circumferential row 302 spaced apart from row 301. In certain embodiments, first and second circumferential rows 301 and 302 can be configured to provide imaging data from different regions. For example, in one embodiment first circumferential row 301 can be configured to provide imaging data from the region of an occlusion, while second circumferential row 302 can be configured to provide imaging data of the lumen (e.g. an artery or vein) into which ultrasonic transceiver 115 is inserted.

Referring now to FIG. 16, transducers 300 are sequentially activated to transmit a signal 310. In the view shown in FIG. 16, a transducer 300 in the middle portion of the array is shown transmitting signal 310 toward a target 328. Reflected signals 320 are directed from target 328 back to transducers 300. Certain embodiments may incorporate aspects of commercially available systems, including for example, the Eagle Eye Platinum digital intravascular ultrasound (IVUS) available from Koninklijke Philips N.V®.

Embodiments incorporating transducers 300 extending around the perimeter of ultrasonic transceiver 115 can provide certain features not found in embodiment incorporating a rotating array of transducers. For example, embodiments incorporating transducers 300 extending around the perimeter of ultrasonic transceiver 115 do not produce guide wire artifacts because the photoacoustic signals are transmitted and received from multiple points around the circumference of transceiver 115. Accordingly, a guidewire would not block the transmission or reception of photoacoustic signals for each of transducers 300 extending around the perimeter of ultrasonic transceiver 115, and would not produce an artifact (in contrast a rotating linear array of transducers). Furthermore, embodiments incorporating circumferential transducers such as those shown in FIGS. 15 and 16 typically do include smaller apertures for the transducers (e.g. less than 1 mm, as compared to approximately 3 mm for rotating array transducer embodiments). In addition, circumferential transducer embodiments can require higher frequency lasers in the KHz range as opposed to rotating transducer embodiments with a laser frequency of approximately 100 Hz.

Limited-view artifacts are commonly present in optoacoustic tomography images, e.g. due to practical geometrical and physical constraints imposed by the imaging systems. Close distance reconstruction provides a higher contrast-noise ratio (CNR), while image reconstruction for opto-acoustic (OA) excitation at longer distances (e.g. greater than 50 mm) provides a lower CNR. This lower CNR may make it difficult to reconstruct an image comprising a full circumference (e.g. of a blood vessel). In addition to relocation of the ultrasonic transceiver to increase the CNR (e.g. by placing ultrasonic transceiver in a vein proximal to the area being investigated), other embodiments may comprise different photoacoustic excitation light transmitter fiber tip geometries. Such geometries can provide for improved CNR at greater OA excitation distances.

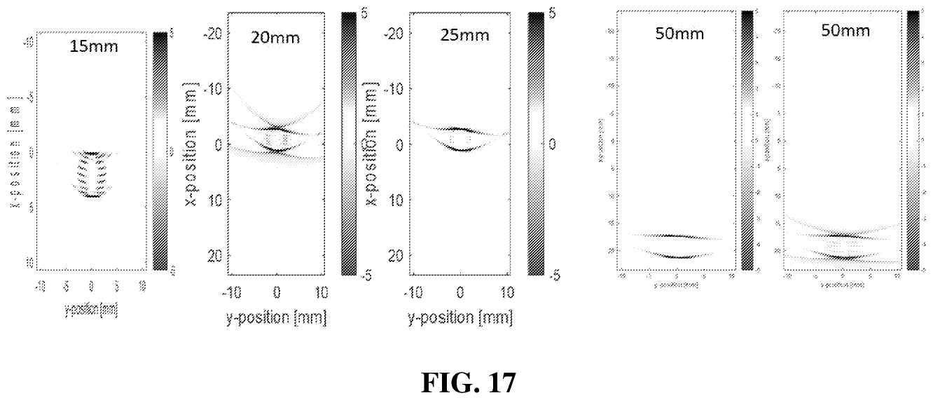

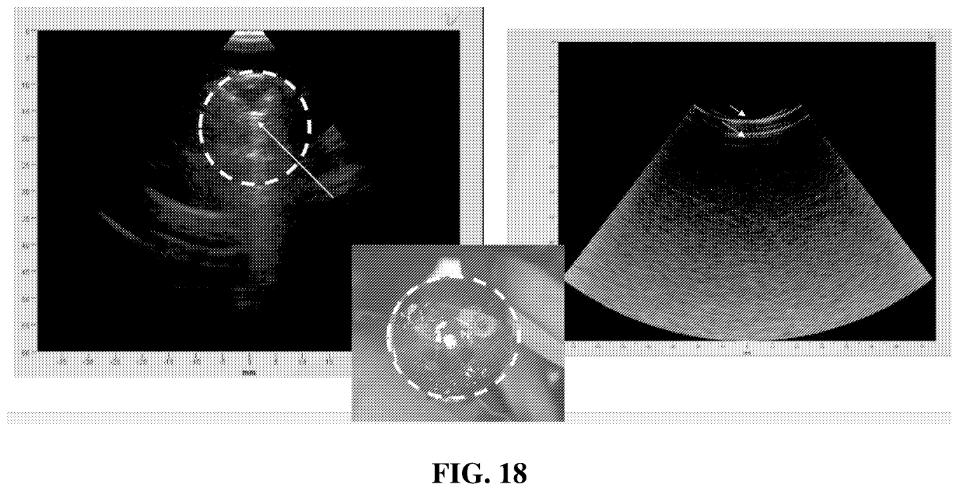

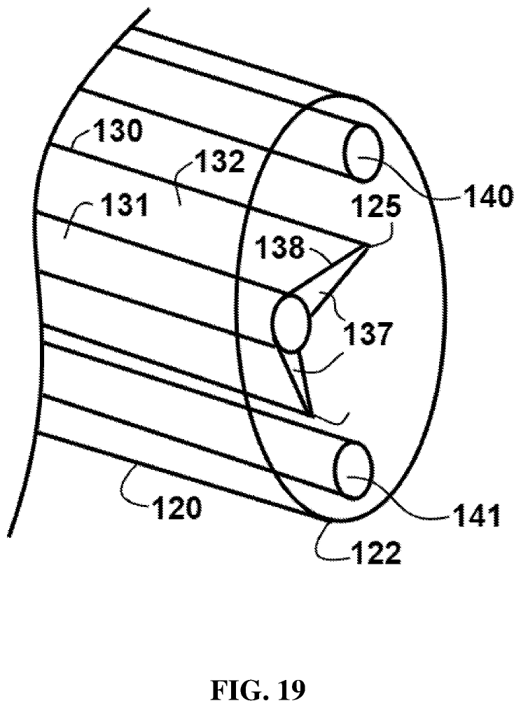

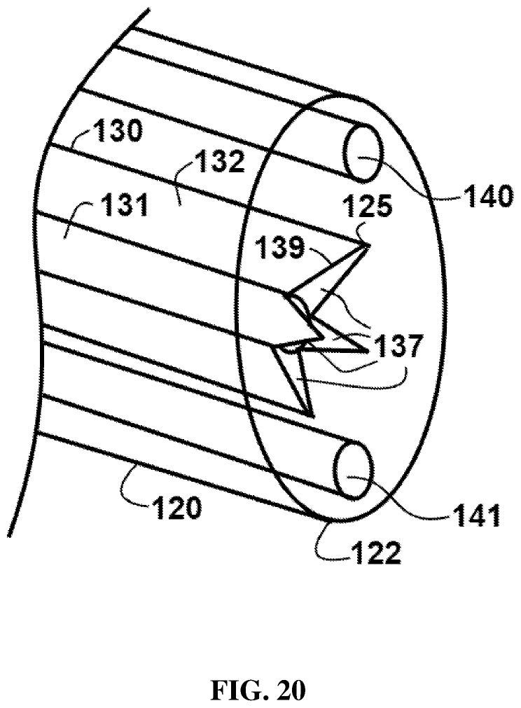

Longer distances can result in a limited view problem in optoacoustic reconstruction. In this scenario, the aperture of the US probe is typically a fraction of the distance from the US probe to the intended target structure to be detected and/or relative size of the intended target of detection. For example, a 3 mm linear array placed 50 mm away from a 3 mm ring, will have difficulty isolating the entire curvature of the ring except for the curvature that is perpendicular to the US probe's aperture. FIG. 17 illustrates a time reversal algorithm shows a degradation of “circle” wall detection with distance in mm from 15 mm to 50 mm. FIG. 18 is an experimental result with US/OA image collected with a 64 element probe using an artery phantom illustrating the effect.

However, by modifying the conical tip of the fiber to focus the light to generate a colleciton of points (slide 3), it is possible to reconstruct all of the curvature of the vessel even at long distance like 50-55 mm. (slide 4). Slide 5 shows a comparison between the two cases of a contiguous (ring) excitation v.s. an excitation that is a collection of points to complete the ring. OA detection may be possible using point excitations done by modifying the tip of the fiber to emit a ring of discrete points or a collection of discrete points.

For example, in addition to the inward and outward conical tips shown in FIGS. 3 and 4, in certain embodiments photoacoustic excitation light transmitter fiber 125 may comprise a multi-faceted tip 138 or 139 as shown in FIGS. 19 and 20, respectively. Multi-faceted tips 138 and 139 comprise a plurality of surfaces 137 that can generate additional output beams from photoacoustic excitation light transmitter 125. For example, multi-faceted tip 138 can generate two output beams from photoacoustic excitation light transmitter 125, while multi-faceted tip 139 can generate four output beams from photoacoustic excitation light transmitter 125. The multiple output beams can generate multiple response signals, which can be analyzed in combination to improve CNR at greater OA excitation distances. Other aspects of FIGS. 19 and 20 (other than the fiber tip geometry) are equivalent to those discussed in FIGS. 3 and 4 and will not be repeated here. Except as otherwise discussed herein, it is understood that the embodiments shown in FIGS. 19 and 20 operate in a manner similar to the previously discussed embodiments.

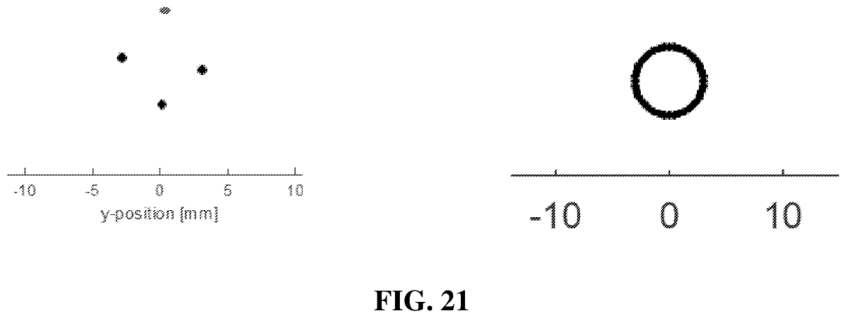

As shown in FIG. 21 by modifying the conical tip of the fiber (e.g. as shown in FIG. 19 or 20) to focus the light to generate a collection of discrete points. By modifying the fiber tip geometry, it is possible to provide a “ring” of point outputs as shown on the left, instead of a contiguous output as shown on the right for a conical tip. In the embodiment shown, four opposing output beams are provided with an adjustable angle between beams can be used to create the output on the left.

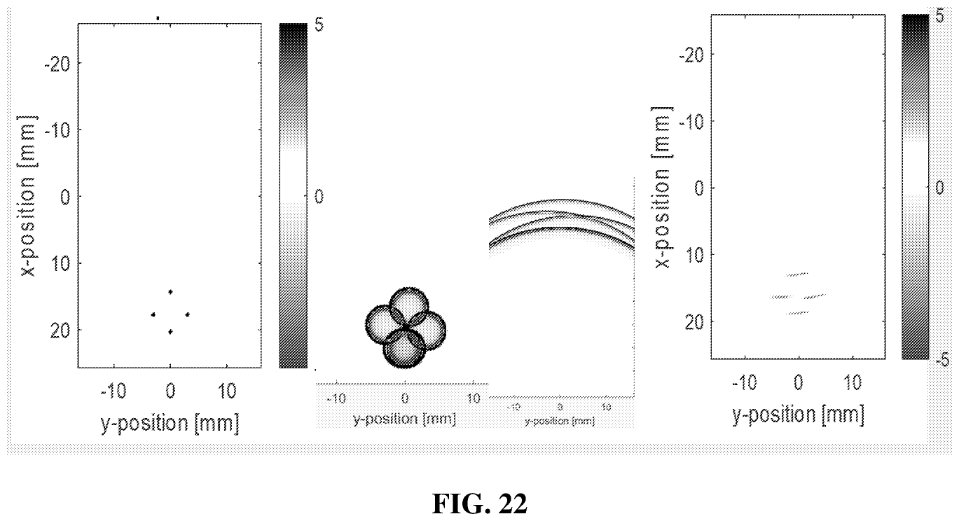

FIG. 22 illustrates a simulated reconstruction that shows the ability to complete all sides of the circle. As shown in FIG. 22, it is possible to reconstruct all of the curvature of the vessel, even at long distances such as 50-55 mm.

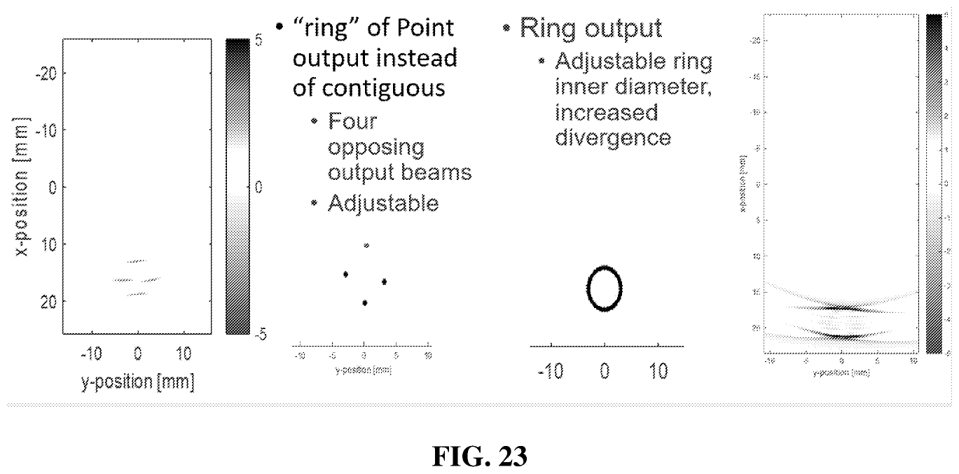

FIG. 23 illustrates outlining of the vessel wall is possible on all sides (instead of just top and bottom). Specifically, FIG. 23 shows a comparison between the two cases of a contiguous (ring) excitation on the right, versus an excitation that is a collection of points to complete the ring on the left.

Results

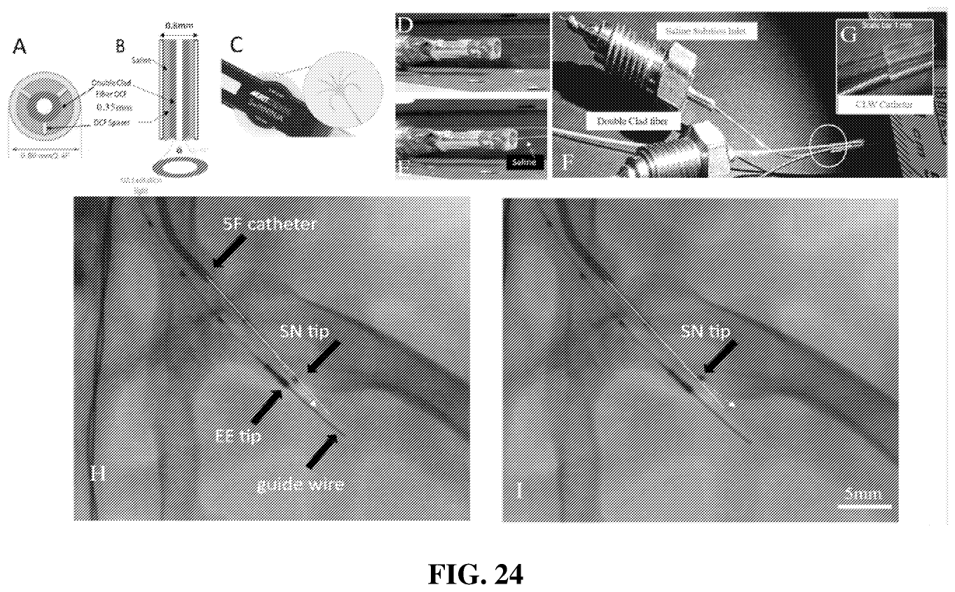

Referring now to FIG. 24, a transvenous imaging approach for a rabbit model is shown according to an exemplary embodiment of the present disclosure. FIG. 24 panel A illustrates a schematic cross-section end view of a SwiftNINJA® showing a double clad fiber (DCF) in a central lumen with a circumferential saline port. Panel B of FIG. 24 illustrates a schematic cross-section side view showing the central lumen with spacer centering the DCF allowing ejection of microliter saline volumes. In this embodiment, the OA excitation light propagates from DCF in ring geometry, and Tm laser light emits from the central lumen directly forward to resect a CTO. As shown in FIG. 24 panel C, this embodiment provides Cold Laser Wire (CLW) steerability via two deflections and translation. Panels D and E illustrate a saline injector valve with fast response time (milliseconds) showing the release of saline with an electronic trigger, while panels F and G illustrate a prototype assembly of the CLW. FIG. 24 panels H and I illustrate the minute movements possible with the SwiftNINJA® (SN) during in vivo rabbit femoral CTO experiments. Markers on the EagleEye™ (EE) placed in the adjacent vein are visible as it is advanced over a guidewire, and radio-opaque markers on the SwitftNINJA (SN) show tip position with respect to the axis of the 5Fr catheter.

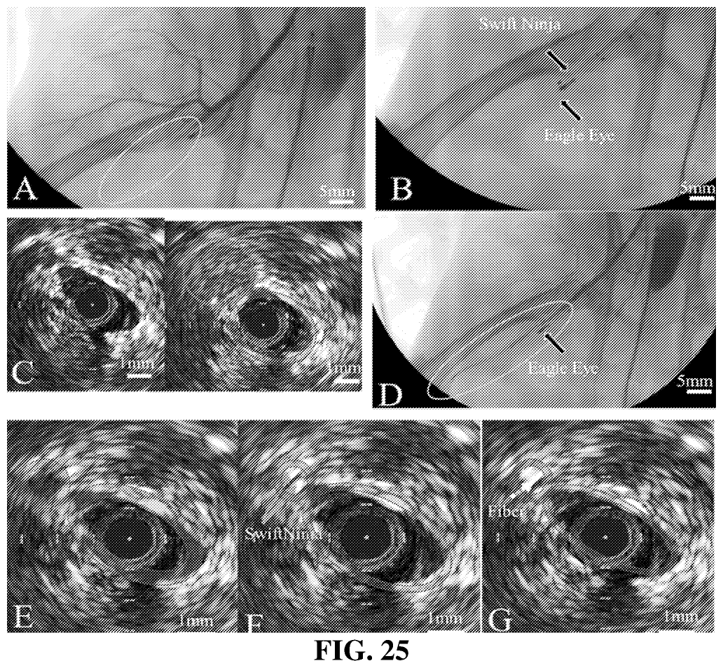

Referring now to FIG. 25, panel A illustrates contrast angiography done before PCI intervention with a CLW showing blockage (indicated by the oval) due to introduction of an acute CTO plug using thrombin. Panel B illustrates placement of the SwiftNINJA® and EagleEye™ in the femoral artery and adjacent vein respectively. Panel C shows confirmation of the artery (upper left circle) using tiny air bubbles injected through the SwiftNINJA© into the proximal region of the femoral artery before CLW crossing. Panel D shows post contrast angiography of the femoral artery (oval) after CLW (SwiftNINJA® plus optical fiber) crossing. Panels E, F and G show periodic images of the artery (upper left circles) through the transvenous imaging approach during the PCI crossing showing the SwiftNINJA® and optical fibner inside the switch during the crossing. The panel views of E,F,G were utilized to guide the direction of the CLW with respect to the true lumen (upper left circle) during PCI crossing.

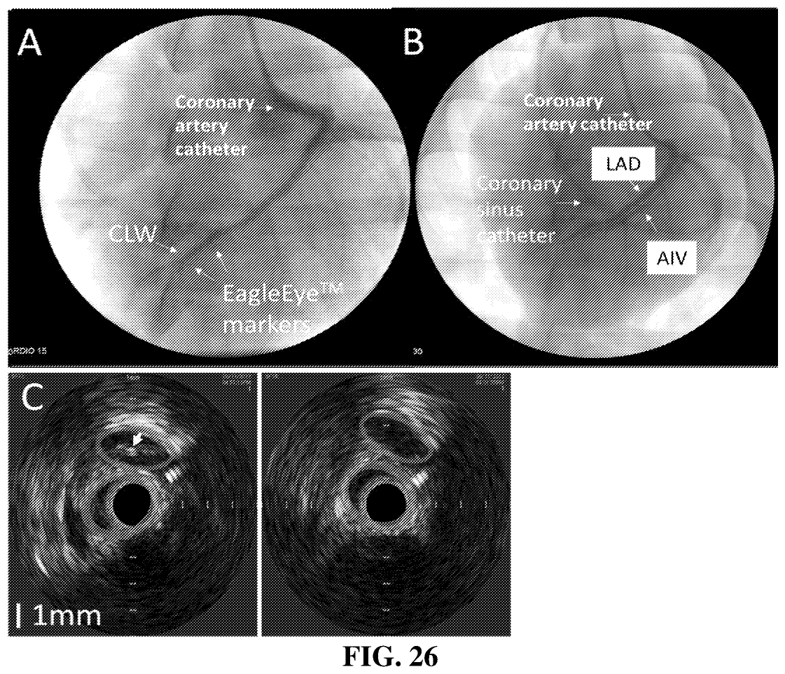

FIG. 26 panels A and B illustrate a demonstration of transvenous catheter placement in an in vivo porcine heart according to an exemplary embodiment of the present disclosure. Panel C illustrates a transvenous (lower circle) EagleEye™ images of CLW fiber tip (arrow) in mid LAD (upper circle) in ex vivo porcine heart

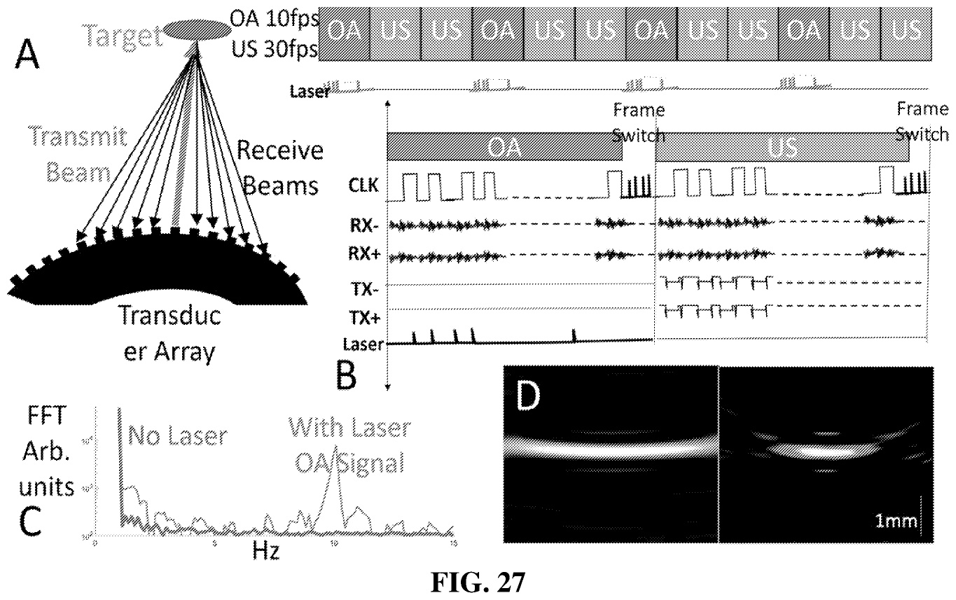

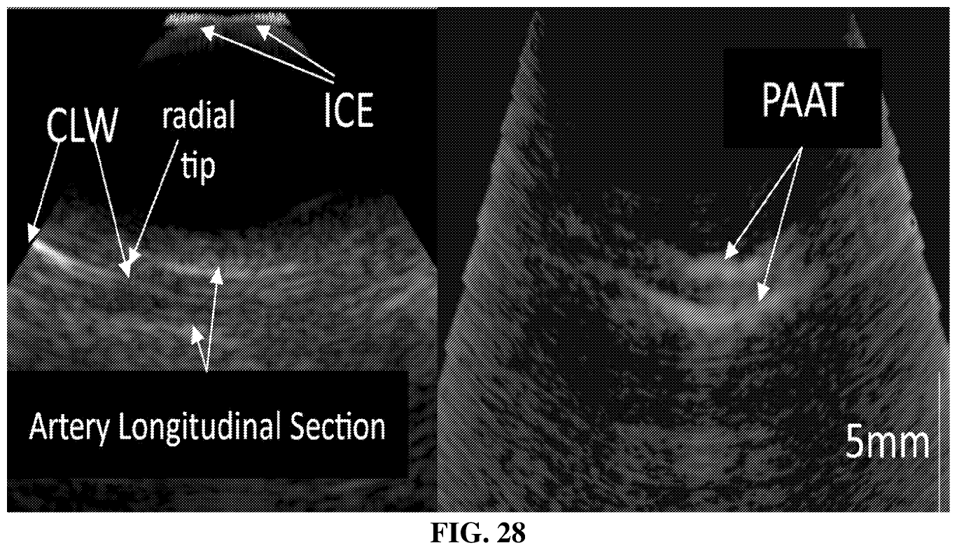

FIG. 27 panel A shows an EagleEye™ electronic scanning scheme, while panel B illustrates an OA/US timing diagram illustrating laser-trigger and OA-signal acquisition events. Panel C shows a fast Fourier transform (FFT) of OA-Beacon signal obtained in a phantom with EagleEye™ catheter (OA source: 1064 nm triggered at 500 Hz synchronous with EagleEye™ and 10 Hz OA frame rate). In this embodiment, panel D shows results of a model-based transvenous reconstruction algorithm (TRA) using simulated EagleEye™ data showing improvement (right) in lateral resolution versus standard back-projection (left).

FIG. 28 shows US (left) and OA (right) images of a porcine heart RCA using a 64 element Abbott ViewFlex™ ICE transducer to detect the vessel wall with radial firing fiber at 1205 nm (OA Amplifier: AMP128, Photosound® Inc, OA: approx.10 mJ/cm²).

All of the devices, systems and/or methods disclosed and claimed herein can be made and executed without undue experimentation in light of the present disclosure. While the devices, systems and methods of this invention have been described in terms of particular embodiments, it will be apparent to those of skill in the art that variations may be applied to the devices, systems and/or methods in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

The contents of the following references are incorporated by reference herein:
U.S. Pat. No. 5,944,687
U.S. Patent Publication 2013/0102865
U.S. Patent Publication 2014/0276015
PCT Patent Publication WO2013/123020
PCT Patent Publication WO2019/097317
[1] L. Azzalini et al., “Epidemiology, Management Strategies, and Outcomes of Patients With Chronic Total Coronary Occlusion,” Am. J. Cardiol., vol. 118, no. 8, pp. 1128-1135, October 2016.
[2] M. Tajstra, “Impact of Chronic Total Occlusion of the Coronary Artery on Long-Term Prognosis in Patients With Ischemic Systolic Heart Failure: Insights From the COMMIT-HF Registry,” JACC Cardiovasc Interv, pp. 9-17, 2016.
[3] J. A. Suero, “Procedural outcomes and long-term survival among patients undergoing percutaneous coronary intervention of a chronic total occlusion in native coronary arteries: A 20-year experience,” J. Am. Coll. Cardiol., pp. 32-38, 2001.
[4] E. S. Brilakis, “Procedural outcomes of chronic total occlusion percutaneous coronary intervention: a report from the NCDR (National Cardiovascular Data Registry),” JACC Cardiovasc Interv, pp. 2-8, 2015.
[5] J. Sapontis, “Health Status Outcomes After Chronic Total Occlusion Angioplasty: A Report From the OPEN-CTO Registry (Outcomes, Patient Health Status, and Efficiency in Chronic Total Occlusion Hybrid Procedures),” JACC Cardiovasc Interv, pp. 10-15, 2017.
[6] R. J. van der Schaaf, “Impact of multivessel coronary disease on long-term mortality in patients with ST-elevation myocardial infarction is due to the presence of a chronic total occlusion,” Am J Cardiol, pp. 98-99, 2006.
[7] C. Godino, “Coronary chronic total occlusions: mid-term comparison of clinical outcome following the use of the guided-STAR technique and conventional anterograde approaches,” Catheter Cardiovasc Interv, pp. 71-79, 2012.
[8] R. Valenti, “Predictors of reocclusion after successful drug-eluting stent-supported percutaneous coronary intervention of chronic total occlusion,” J Am Coll Cardiol, pp. 61-65, 2013.
[9] G. E. Christakopoulos, “Meta-Analysis of Clinical Outcomes of Patients Who Underwent Percutaneous Coronary Interventions for Chronic Total Occlusions,” Am.

J. Cardiol., pp. 110-115, 2015.
[10] G. Alessandrino, “A Clinical and Angiographic Scoring System to Predict the Probability of Successful First-Attempt Percutaneous Coronary Intervention in Patients With Total Chronic Coronary Occlusion,” JACC Cardiovasc Interv, pp. 8-12, 2015.
[11] N. Lo, “Periprocedural myocardial injury in chronic total occlusion percutaneous interventions: a systematic cardiac biomarker evaluation study,” JACC Cardiovasc Interv, pp. 1-7, 2014.
[12] J. D. Abbott et al., “Recent Trends in the Percutaneous Treatment of Chronic Total Coronary Occlusions,” Am. J. Cardiol., vol. 97, no. 12, pp. 1691-1696, June 2006.
[13] A. Colombo et al., “Treating chronic total occlusions using subintimal tracking and reentry: The STAR technique,” Catheter. Cardiovasc. Interv., vol. 64, no. 4, pp. 407-411, 2005.
[14] Z. M. Hijazi, K. Shivkumar, and D. J. Sahn, “Intracardiac Echocardiography During Interventional and Electrophysiological Cardiac Catheterization,” Circ. Cardiovasc. Interv., vol. 119, no. 4, pp. 587-596, 2009.
[15] A. S. Cardiac and B. Evaluation, “Periprocedural Myocardial Injury in Chronic Total Occlusion Percutaneous Interventions,” JACC Cardiovasc. Interv., vol. 7, no. 1, pp. 47-54, 2014.
[16] L. Song et al., “Intravascular Ultrasound Analysis of Intraplaque Versus Subintimal Tracking in Percutaneous Intervention for Coronary Chronic Total Occlusions and Association With Procedural Outcomes,” JACC Cardiovasc. Interv., vol. 10, no. 10, pp. 1011-1021, 2017.
[17] P. Tajti et al., “The Hybrid Approach to Chronic Total Occlusion Percutaneous Coronary Intervention,” JACC Cardiovasc. Interv., vol. 11, no. 14, pp. 1325-1335, 2018.
[18] C. Godino et al., “Original Studies Coronary Chronic Total Occlusions: Mid-Term Comparison of Clinical Outcome Following the Use of the Guided-STAR Technique and Conventional Anterograde Approaches,” Catheter. Cardiovasc. Interv., vol. 79, pp. 20-27, 2012.
[19] S. S. Naidu et al., “SCAI Expert Consensus Statement: 2016 Best Practices in the Cardiac Catheterization Laboratory: (Endorsed by the Cardiological Society of India, and Sociedad Latino Americana de Cardiologia Intervencionista; Affirmation of Value by the Canadian Associatio,” Catheter. Cardiovasc. Interv., vol. 88, pp. 1-6, 2016.
[20] D. A. Jones et al., “Successful recanalization of chronic total occlusions is associated with improved long-term survival,” JACC Cardiovasc. Interv., vol. 5, no. 4, pp. 380-388, 2012.
[21] A. Sakes, E. Regar, J. Dankelman, and P. Breedveld, “Crossing Total Occlusions: Navigating Towards Recanalization,” Cardiovasc. Eng. Technol., vol. 7, no. 2, pp. 103-117, 2016.
[22] O. Topaz et al., “Acute results, complications, and effect of lesion characteristics on outcome with the solid-state, pulsed-wave, mid-infrared laser angioplasty system: Final multicenter registry report,” Lasers Surg. Med., vol. 22, no. 4, pp. 228-239, 1998.
[23] G. Fagogenis et al., “Autonomous robotic intracardiac catheter navigation using haptic vision,” Sci. Robot., vol. 4, no. 29, 2019.
[24] “Robocath Successfully Completes First Robotic Coronary Angioplasties in Germany With R-One,” CathLab Digest, 2020. [Online]. Available: https://www.cathlabdigest.com/content/robocath-successfully-completes-first-robotic-coronary-angioplasties-germany-r-one.
[25] B. Zhu, “Development of a total atherosclerotic occlusion with cell-mediated calcium deposits in a rabbit femoral artery using tissue-engineering scaffolds,” J Tissue Eng Regen Med, pp. 3-6, 2012.
[26] N. Nili, “Unexpected calcification after direct intravascular injection of autologous bone marrow-derived cells in a chronic total occlusion model,” EuroIntervention, pp. 10-13, 2014.
[27] F. Ikeno, Y. Suzuki, and A. C. Yeung, “The representative porcine model for human cardiovascular disease,” J. Biomed. Biotechnol., vol. 2011, 2011.
[28] P. Fefer et al., “Characterisation of a novel porcine coronary artery CTO model,” EuroIntervention, vol. 7, no. 12, pp. 1444-1452, April 2012.
[29] R. H. Goetz, M. Rohman, J. D. Haller, R. Dee, and S. S. Rosenak, “Internal mammary-coronary artery anastomosis. A nonsuture method employing tantalum rings.,” J. Thorac. Cardiovasc. Surg., vol. 41, pp. 378-386, March 1961.
[30] E. R. J. A. Lles et al., “Video-rate all-optical ultrasound imaging,” Biomed. Opt. Express, vol. 9, no. 8, pp. 3481-3494, 2018.
[31] G. Wissmeyer, M. A. Pleitez, A. Rosenthal, and V. Ntziachristos, “Looking at sound: optoacoustics with all-optical ultrasound detection,” Light Sci. Appl., vol. 7, no. 1, 2018.
[32] J. A. Guggenheim et al., “Ultrasensitive plano-concave optical microresonators for ultrasound sensing,” Nat. Photonics, vol. 11, no. 11, pp. 714-719, 2017.
[33] J. L. Su, B. Wang, and S. Y. Emelianov, “Spectroscopic intravascular photoacoustic imaging of neovasculature: phantom studies,” 2009, vol. 7177, no., p. 717727.
[34] G. P. Luke, D. Yeager, and S. Y. Emelianov, “Biomedical applications of photoacoustic imaging with exogenous contrast agents,” Ann. Biomed. Eng., vol. 40, no. 2, pp. 422-437, 2012.
[35] K. Jansen, M. Wu, A. F. W. Van Der Steen, and G. Van Soest, “Lipid detection in atherosclerotic human coronaries by spectroscopic intravascular photoacoustic imaging,” vol. 21, no. 18, pp. 2200-2206, 2013.
[36] K. Jansen, M. Wu, A. F. W. Van Der Steen, and G. Van Soest, “Photoacoustic imaging of human

coronary atherosclerosis in two spectral bands,” Biochem. Pharmacol., vol. 2, no. 1, pp. 12-20, 2014.
[37] M. Wu et al., “Real-time volumetric lipid imaging in vivo by intravascular photoacoustics at 20 frames per second,” Biomed. Opt. Express, vol. 8, no. 2, p. 943, 2017.
[38] K. Jansen, G. van Soest, and A. F. W. van der Steen, “Intravascular Photoacoustic Imaging: A New Tool for Vulnerable Plaque Identification,” Ultrasound Med. Biol., vol. 40, no. 6, pp. 1037-1048, 2014.
[39] Y. Kim, M. Gnanadesigan, G. Van Soest, and T. W. Johnson, “A new technique for lipid core plaque detection by optical coherence tomography for prevention of periprocedural myocardial infarction,” vol. 23, no. April, pp. 10-12, 2017.
[40] L. V Wang, S. Hu, L. V Wang, and S. Hu, “Photoacoustic Tomography: In Vivo Imaging from Organelles to Organs Linked references are available on JSTOR for this article: Photoacoustic Tomography: In Vivo Imaging from Organelles to Organs,” Science (80-.)., vol. 335, no. 6075, pp. 1458-1462, 2012.
[41] G. J. Vassar et al., “Holmium: YAG Lithotripsy: Photothermal Mechanism,” J. Endourol., vol. 13, no. 3, pp. 181-190, April 1999.
[42] T. Wang et al., “Detection of plaque structure and composition using OCT combined with two-photon luminescence (TPL) imaging,” Lasers Surg. Med., vol. 47, no. 6, pp. 485-494, 2015.
[43] T. Wang et al., “Combined two-photon luminescence microscopy and OCT for macrophage detection in the hypercholesterolemic rabbit aorta using plasmonic gold nanorose,” Lasers Surg. Med., vol. 44, no. 1, pp. 49-59, January 2012.
[44] T. Wang et al., “Dual-modality fiber-based OCT-TPL imaging system for simultaneous microstructural and molecular analysis of atherosclerotic plaques,” Biomed. Opt. Express, vol. 6, no. 5, pp. 1665-1678, 2015.
[45] G. J. Reis, “Laser balloon angioplasty: clinical, angiographic and histologic results,” J Am Coll Cardiol, pp. 11-18, 1991.
[46] K. J. Cho, “Carbon Dioxide Angiography: Scientific Principles and Practice,” Vasc.

Spec. Int., vol. 31, no. 3, 2015.
[47] R. R. Anderson et al., “Selective photothermolysis of lipid-rich tissues: A free electron laser study,” Lasers Surg. Med., vol. 38, no. 10, pp. 913-919, December 2006.

1. An apparatus configured for guidance for treatment of a chronic total occlusion, the apparatus comprising:
a first catheter, wherein the first catheter comprises an ultrasound transceiver; and
a second catheter, wherein the second catheter comprises a proximal end and a distal end; and
a photoacoustic excitation light transmitter positioned at the distal end of the second catheter, wherein:
the photoacoustic excitation light transmitter emits excitation light in a conical pattern;
the photoacoustic excitation light transmitter emits excitation light at a pulse duration between 50 femtoseconds (fs) and 1 microsecond (μs); and
the second catheter configured to detect photoacoustic signals resulting from the absorption of excitation light emitted by the photoacoustic excitation light transmitter.

2. The apparatus of claim 1 further comprising a control module, wherein the control module is coupled to the first catheter and the second catheter.

3. The apparatus of claim 1 wherein the ultrasound transceiver is configured as a phased array.

4. The apparatus of claim 1 wherein the ultrasound transceiver comprises a plurality of transducers arranged in a circumferential row extending around the ultrasound transceiver.

5. The apparatus of claim 4 wherein the circumferential row is a first circumferential row, and wherein the plurality of transducers are further arranged in a second circumferential row extending around the ultrasound transceiver.

6. The apparatus of claim 2 wherein the control module is configured to control the pulse duration of the excitation light.

7. The apparatus of claim 1 wherein the second catheter comprises a photonic crystal fiber.

8. The apparatus of claim 7 wherein the photonic crystal fiber is a double clad photonic crystal fiber.

9. The apparatus of claim 8 wherein:
the double clad fiber comprises a core and a cladding; and
the photoacoustic excitation light transmitter is configured as a conical tip of the cladding at the distal end of the second catheter.

10. The apparatus of claim 9 wherein the conical tip extends outward from the distal end.

11. The apparatus of claim 9 wherein the conical tip extends inward from the distal end.

12. The apparatus of claim 9 wherein the core is configured to provide illumination for close-range imaging of a region directly in front of the distal end.

13. The apparatus of claim 7 wherein the photoacoustic excitation light transmitter is configured as a conical tip of the photonic crystal fiber at the distal end of the second catheter.

14. The apparatus of claim 7 wherein the photonic crystal fiber comprises a multi-faceted tip.

15. The apparatus of claim 1 wherein the second catheter is configured to emit excitation light at a wavelength of 930 nanometers (nm).

16. The apparatus of claim 1 wherein the second catheter is configured to emit excitation light at a wavelength between 1200 nm and 1240 nm.

17. The apparatus of claim 1 wherein the second catheter is configured to emit excitation light at a wavelength of 1210 nm.

18. The apparatus of claim 1 wherein the second catheter is configured to emit excitation light at a wavelength between 1700 nm and 1740 nm.

19. The apparatus of claim 1 wherein the second catheter is configured to emit excitation light at a wavelength of 1720 nm.

20. The apparatus of claim 1 wherein the second catheter is configured to emit excitation light at a first wavelength that is lipid-specific and a second wavelength that is blood-specific.

21. The apparatus of claim 1 wherein the second catheter is configured to emit excitation light at a first wavelength of 915 nm, 1210 nm, or 1720 nm and a second wavelength of 532 nm, 980 nm, or 808 nm.

22. A method of imaging a blood vessel containing a chronic total occlusion (CTO), the method comprising:
directing a first catheter into a region of a heart, wherein the first catheter comprises an ultrasound transceiver;
directing a second catheter into an artery comprising a chronic total occlusion (CTO);
emitting photoacoustic excitation light from a distal end of the second catheter, wherein:
the photoacoustic excitation light is emitted in a conical pattern;
the photoacoustic excitation light is emitted at a pulse duration between 50 fs and 1 us; and
the photoacoustic excitation light generates a photoacoustic signal by light absorption in tissues surrounding the artery or in tissue in the CTO; and
detecting the photoacoustic signal emitted from the periphery of the artery or the CTO via first catheter.

23. The method of claim 22 wherein:
the artery is a right coronary artery; and
the region of the heart where the first catheter is directed is a right atrium proximal to the right coronary artery.

24. The method of claim 22 wherein:
the artery is a left anterior descending artery; and
the region of the heart where the first catheter is directed is a right ventricle proximal to the left anterior descending artery.

25. The method of claim 22 wherein:
the artery is a left anterior descending artery; and
the region of the heart where the first catheter is directed is a vein proximal to the left anterior descending artery.

26. The method of claim 22 wherein:
the artery is a left circumflex artery; and
the region of the heart where the first catheter is directed is a right ventricle proximal to the left anterior circumflex artery.

27. The method of claim 22 wherein:
the artery is a left circumflex artery; and
the region of the heart where the first catheter is directed is a vein proximal to the left anterior circumflex artery.

28. The method of claim 22 wherein:
the artery is a left anterior descending artery; and
the region of the heart where the first catheter is directed is a left ventricle proximal to the left anterior descending artery.

29. The method of claim 22 wherein:
the artery is a left circumflex artery; and
the region of the heart where the first catheter is directed is a left ventricle proximal to the left anterior circumflex artery.

30. The method of claim 22 wherein the photoacoustic excitation light is emitted at a wavelength of 930 nanometers (nm).

31. The method of claim 22 wherein the second catheter is configured to emit excitation light at a wavelength between 1200 nm and 1240 nm.

32. The method of claim 22 wherein the second catheter is configured to emit excitation light at a wavelength of 1210 nm.

33. The method of claim 22 wherein the second catheter is configured to emit excitation light at a wavelength between 1700 nm and 1740 nm.

34. The method of claim 22 wherein the second catheter is configured to emit excitation light at a wavelength of 1720 nm.

35. The method of claim 22, further comprising:
emitting a transmitted ultrasonic signal from the ultrasound transceiver; and
receiving a remitted ultrasonic signal by the ultrasound transceiver.

36. The method of claim 35 wherein the photoacoustic signal and the remitted ultrasonic signal are utilized to kinematically direct the second catheter.

37. The method of claim 36 wherein the kinematic direction may comprise any combination of mechanical translation or re-orientation.

38. The method of claim 22, wherein:
the first catheter and the second catheter are coupled to a control module; and
the control module is configured to control the pulse duration of the excitation light.

39. The method of claim 22 wherein:
the second catheter comprises a photonic crystal fiber; and
the photoacoustic excitation light is emitted from the photonic crystal fiber.

40. The method of claim 39 wherein the photonic crystal fiber is a double clad photonic crystal fiber.

41. The method of claim 40 wherein:
the double clad fiber comprises a core and a cladding; and
the photoacoustic excitation light is emitted from a conical tip of the cladding at the distal end of the second catheter.

42. The method of claim 41 wherein the conical tip extends outward from the distal end of the second catheter.

43. The method of claim 41 wherein the conical tip extends inward from the distal end of the second catheter.

44. The method of claim 9 further comprising illuminating a region directly in front of the distal end of the second catheter via the core of the double clad fiber.
